(19) United States
(12) Patent Application Publication
(10) Pub. No.: US 2025/0177413 A1
(43) Pub. Date: Jun. 5, 2025

(54) SHORT ACTING ESTERS OF IVABRADINE

(71) Applicant: Academic Pharmaceuticals, INC., Lake Bluff, IL (US)

(72) Inventor: John Charin Somberg, Lake Forest, IL (US)

(21) Appl. No.: 18/963,877

(22) Filed: Nov. 29, 2024

Related U.S. Application Data
(60) Provisional application No. 63/604,486, filed on Nov. 30, 2023.

(51) Int. Cl. A61K 31/55; C07D 223/16

(52) U.S. Cl. CPC A61K 31/55; C07D 223/16

(57) ABSTRACT

Some aspects provide novel ivabradine-like compounds and pharmaceutical compositions containing the same that are useful as pharmaceutical agents in the reduction of heart rate without a concomitant clinically significant fall in blood pressure with rapid termination of HR effect when stopping administration.

### FIELD OF THE INVENTION

Herein are described novel ivabradine compounds and pharmaceutical compositions containing the same. The compounds and compositions are useful as pharmaceutical agents for lowering a patient's heart rate without concomitant blood pressure reduction with a short duration of action.

### BACKGROUND OF THE INVENTION

The availability of an ultra-short acting heart rate (HR) reducing agent would have great benefit especially since ivabradine esters would only reduce HR without a direct blood pressure (BP) action while possessing a short duration of action. As an example, in surgery and especially during the initial induction of anesthesia, HR may dramatically increase, increasing O₂ consumption which can be deleterious and in the patient with coronary artery disease cause an O₂/demand imbalance precipitating a myocardial infarction. Often anesthesiologists utilize esmolol to modulate HR. However, esmolol also lowers BP and this is very problematic in patients with an already low BP or who develop low BP during surgery due to blood loss or third spacing of fluid. A pharmaceutical that could lower HR without a major effect on BP that was short acting would be an ideal agent in many situations especially in operative patients. However, ivabradine is problematic in that once given its effects last for hours. During the course of surgery and into recovery an increase in HR and thus cardiac output may be physiologically a necessity. Ivabradine reduction in HR would not have dissipated and thus the initial benefits lost. An ivabradine like drug that has a short time to dissipate its HR lowering action would be far more salutary.

There are other conditions and situations where a selective HR reducing agent that is short-acting and does not affect BP would be useful. Patients in intensive care settings with sinus tachycardia utilize excessive O₂ and reducing HR without a BP effect would be salutary. Patients undergoing CT angiography (CTA) need to have a slow constant HR (90 bpm or less) during the procedure without BP lowering, which can decrease coronary perfusion. A short duration of HR slowing without BP effect would be ideal and thus another use for a short-acting HR reducing agent.

Additionally, the I_{f} channels are found at the atrial ventricular junction (AV junction) in the AV node. This site is the junction where conduction occurs from the atrium to ventricles. In pediatric patients who are undergoing cardiac surgery, usually correction of congenital cardiac abnormalities, patients can develop a condition of junctural ectopic tachycardia (JET) that can lead to hemodynamic collapse and possibly death. The JET arrhythmia is most difficult to control. Recent studies have reported that ivabradine may be effective treatment, but its duration of action may be problematic after initial arrhythmia control.

The introduction of a short acting ivabradine-like drug would offer considerable advantage for the temporary reduction of HR. Therefore, it is desirable to discover ivabradine-like compounds (e.g., esivabradine) that metabolize faster than ivabradine thus offering a rapid onset of action and a rapid offset of action.

### SUMMARY OF THE INVENTION

Accordingly, in some aspects, there are described novel compounds and stereoisomers or pharmaceutically acceptable salts thereof.

In some aspects, there are described novel pharmaceutical compositions, comprising: a pharmaceutically acceptable carrier and a therapeutically effective amount of at least one of the compounds described herein or a stereoisomer or pharmaceutically acceptable salt thereof.

In some aspects, there are described novel methods of treating various therapeutic indications of esters of ivabradine by administering to a subject a therapeutically effective amount of a compound described herein.

In some aspects, there are described compounds for use in medical therapy.

In some aspects, there is described the use of compounds described herein for the manufacture of a medicament for the reduction of HR without concomitant BP reduction with a short duration of action.

These and other objects, which will become apparent during the following detailed description, have been achieved by the inventors' discovery that the presently claimed compounds or stereoisomers or pharmaceutically acceptable salt forms thereof are expected to be effective as therapeutic agents.

### DETAILED DESCRIPTION OF THE INVENTION

All references cited herein are hereby incorporated in their entirety herein by reference.

Ivabradine, shown below, is an inhibitor of the I_{f} channel that is found predominantly in the sino atrial (SA) node of the heart.

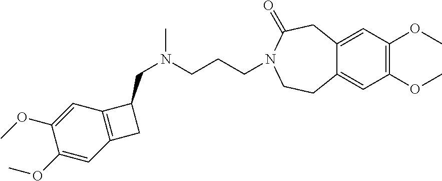

The I_{f} channel is an ultra-fast Na channel that initiates depolarization of the SA node in the heart which is the initiator of cardiac depolarization. This depolarization results in the electrical initiation of cardiac contraction. Ivabradine has been shown to reduce heart rate (HR) in man though the drug possesses a long half-life. It is known that the body has an extensive amount of circulatory esterase's that break down esters in the circulation. By introducing one or more ester linkages into the ivabradine molecule, it is expected that the half-life of ivabradine will be shortened thereby providing HR reduction lasting only minutes instead of the current effective half-life of 6 hours or more in man.

As a result, in some aspects, there is described a novel compound that is expected to be metabolized by human esterases faster than ivabradine (˜6 hours for effective half-life). This novel agent is expected to be rapidly metabolized to reduce the time of the desired activity of the administered agent and thereby minimize the length of time the HR is reduced compared to that observed with ivabradine.

In some aspects, rapid metabolization is achieved by introducing one or more esters into ivabradine to increase its metabolism by esterases and thereby reduce its half-life to be less than that of unmodified ivabradine.

In some aspects, there is described a novel compound of Formula I or a stereoisomer or pharmaceutically acceptable salt thereof:

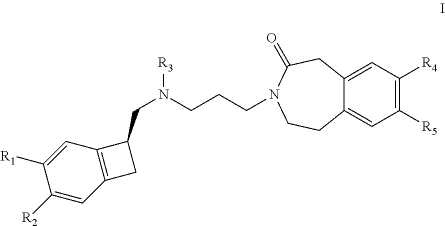

wherein:
at least one of R₁, R₂, R₃, R₄, and R₅ is —CO₂R_{A};
the remainder of R₁, R₂, R₄, and R₅ are independently selected from: OH, OC₁₋₆ alkyl, OCH₂C₆H₅, and OCOCH₃;
R_{A} is independently selected from: H, C₁₋₆ alkyl, C₃₋₆ alkenyl, and C₃₋₆ alkynyl; and,
R₃, if not CO₂R_{A}, is selected from: H, C₁₋₆ alkyl, —CH₂C₆H₅, and —COCH₃.

In some aspects, there are described novel compounds of Formula I or a stereoisomer or a pharmaceutically acceptable salt thereof, wherein:
at least one of R₁, R₂, R₃, R₄, and R₅ is —CO₂R_{A};
the remainder of R₁, R₂, R₄, and R₅ are OCH₃;
R_{A} is independently selected from: H and CH₃;
R₃, if not CO₂R_{A}, is CH₃.

In some aspects, there are described novel compounds of Formula I or a stereoisomer or a pharmaceutically acceptable salt thereof, wherein:
R_{A} is independently CH₃;

In some aspects, there are described novel compounds of Formula I or a stereoisomer or a pharmaceutically acceptable salt thereof, wherein:
at least two of R₁, R₂, R₃, R₄, and R₅ are —CO₂R_{A};
the remainder of R₁, R₂, R₄, and R₅ are OCH₃;
R_{A} is independently selected from: H and CH₃;
R₃, if not CO₂R_{A}, is CH₃.

In some aspects, there are described novel compounds of Formula I or a stereoisomer or a pharmaceutically acceptable salt thereof, wherein:
at least three of R₁, R₂, R₃, R₄, and R₅ are —CO₂R_{A};
the remainder of R₁, R₂, R₄, and R₅ are OCH₃;
R_{A} is independently selected from: H and CH₃;
R₃, if not CO₂R_{A}, is CH₃.

In some aspects, there are described novel compounds of Formula I or a stereoisomer or a pharmaceutically acceptable salt thereof, wherein:
at least four of R₁, R₂, R₃, R₄, and R₅ are —CO₂R_{A};
the remainder of R₁, R₂, R₄, and R₅ are OCH₃;
R_{A} is independently selected from: H and CH₃;
R₃, if not CO₂R_{A}, is CH₃.

In some aspects, there are described novel compounds of Formula I or a stereoisomer or a pharmaceutically acceptable salt thereof, wherein:
R₁, R₂, R₃, R₄, and R₅ are all —CO₂R_{A}; and,
R_{A} is independently selected from: H and CH₃.

In some aspects, there are described novel compounds of Formula IA or a stereoisomer or a pharmaceutically acceptable salt thereof:

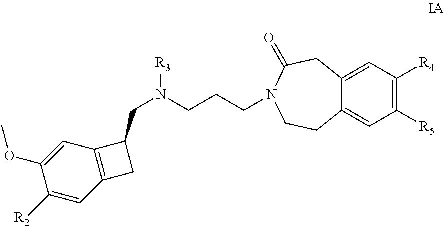

at least one of R₂, R₃, R₄, and R₅ is —CO₂R_{A};
the remainder of R₂, R₄, and R₅ are OCH₃;
R_{A} is independently selected from: H and CH₃;
R₃, if not CO₂R_{A}, is CH₃.

In some aspects, there are described novel compounds of Formula IA₁ or a stereoisomer or a pharmaceutically acceptable salt thereof:

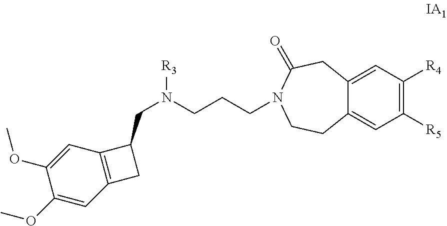

at least one of R₃, R₄, and R₅ is —CO₂R_{A};
the remainder of R₄, and R₅ are OCH₃;
R_{A} is independently selected from: H and CH₃;
R₃, if not CO₂R_{A}, is CH₃.

In some aspects, there are described novel compounds of Formula IB or a stereoisomer or a pharmaceutically acceptable salt thereof, wherein:

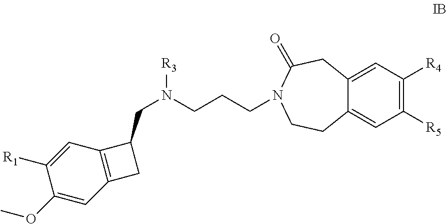

at least one of R₁, R₃, R₄, and R₅ is —CO₂R_{A};
the remainder of R₁, R₄, and R₅ are OCH₃;
R_{A} is independently selected from: H and CH₃;
R₃, if not CO₂R_{A}, is CH₃.

In some aspects, there are described novel compounds of Formula IC or a stereoisomer or a pharmaceutically acceptable salt thereof, wherein:

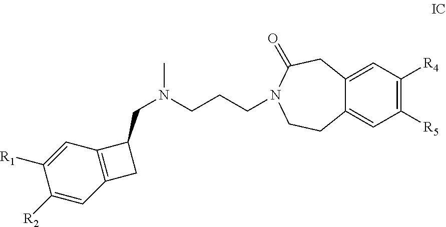

at least one of R₁, R₂, R₄, and R₅ is —CO₂R_{A};
the remainder of R₁, R₂, R₄, and R₅ are OCH₃; and,
R_{A} is independently selected from: H and CH₃.

In some aspects, there are described novel compounds of Formula ID or a stereoisomer or a pharmaceutically acceptable salt thereof, wherein:

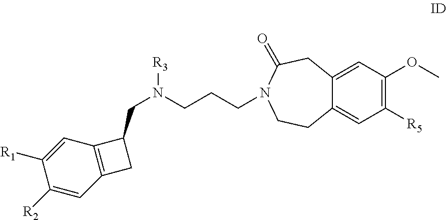

at least one of R₁, R₂, R₃, and R₅ is —CO₂R_{A};
the remainder of R₁, R₂, and R₅ are OCH₃;
R_{A} is independently selected from: H and CH₃;
R₃, if not CO₂R_{A}, is CH₃.

In some aspects, there are described novel compounds of Formula ID₁ or a stereoisomer or a pharmaceutically acceptable salt thereof, wherein:

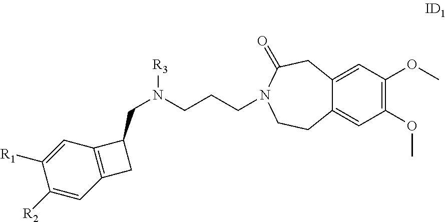

at least one of R₁, R₂, and R₃ is —CO₂R_{A};
the remainder of R₁ and R₂ are OCH₃;
R_{A} is independently selected from: H and CH₃;
R₃, if not CO₂R_{A}, is CH₃.

In some aspects, there are described novel compounds of Formula IE or a stereoisomer or a pharmaceutically acceptable salt thereof, wherein:

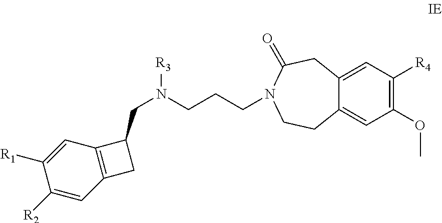

at least one of R₁, R₂, R₃, and R₄ is —CO₂R_{A};
the remainder of R₁, R₂, and R₄ are OCH₃;
R_{A} is independently selected from: H and CH₃;
R₃, if not CO₂R_{A}, is CH₃.

In some aspects, there are described novel compounds of Formula IA, IA₁, IB, IC, ID, ID₁, or IE or a stereoisomer or a pharmaceutically acceptable salt thereof, wherein:
at least two of R₁, R₂, R₃, R₄, and R₅ that are present are —CO₂R_{A};
the remainder of R₁, R₂, R₄, and R₅ are OCH₃;
R_{A} is independently selected from: H and CH₃; and,
R₃, if present and not CO₂R_{A}, is CH₃.

In some aspects, there are described novel compounds of Formula IA, IA₁, IB, IC, ID, ID₁, or IE or a stereoisomer or a pharmaceutically acceptable salt thereof, wherein:
at least two of R₁, R₂, R₃, R₄, and R₅ that are present are —CO₂R_{A};
the remainder of R₁, R₂, R₄, and R₅ are OCH₃;
R_{A} is independently CH₃; and,
R₃, if present and not CO₂R_{A}, is CH₃.

In some aspects, there are described novel compounds of Formula IA, IA₁, IB, IC, ID, ID₁, or IE or a stereoisomer or a pharmaceutically acceptable salt thereof, wherein:
at least three of R₁, R₂, R₃, R₄, and R₅ that are present are —CO₂R_{A};
the remainder of R₁, R₂, R₄, and R₅ are OCH₃;
R_{A} is independently selected from: H and CH₃; and,
R₃, if present and not CO₂R_{A}, is CH₃.

In some aspects, there are described novel compounds of Formula IA, IA₁, IB, IC, ID, ID₁, or IE or a stereoisomer or a pharmaceutically acceptable salt thereof, wherein:
at least three of R₁, R₂, R₃, R₄, and R₅ that are present are —CO₂R_{A};
the remainder of R₁, R₂, R₄, and R₅ are OCH₃;
R_{A} is independently CH₃; and,
R₃, if present and not CO₂R_{A}, is CH₃.

In some aspects, there are described a novel compound of Formula I, or a stereoisomer or pharmaceutically acceptable salt thereof, wherein: one or more hydrogens (H) are replaced by D. For example, R_{A} can be a D, a deuterated methyl group (e.g., CD₃) or the alkenyl or alkynyl hydrogens can be replaced by deuterium (e.g., —CD=CD-). In addition, wherever hydrogens are present in one of the Formulae or in one of the listed substituents (R¹, R², etc.), the hydrogens present (e.g., —CH₂—, alkyl, alkenyl, alkynyl, etc.) can be partially or fully replaced by D (e.g., —CD₂-, CD₃, etc.).

In some aspects, there are described a novel pharmaceutical compositions, comprising: a therapeutically effective amount of a compound described herein or a stereoisomer or a pharmaceutically acceptable salt thereof and a pharmaceutically acceptable carrier.

In some aspects, there are described novel compounds having an effective half-life less than 6 hours (recall that the effective half-life of ivabradine is ˜6 hours). Additional examples include less than 5, 4, 3, 2, and 1 hour. Further examples include less than 60, 50, 40, 30, 20, and 10 minutes. Effective half-life is the time it takes for the activity of ivabradine to be reduced by 50%.

With the compounds described herein being short acting, they offer the advantage of a superior therapeutic to toxic ratio. In another aspect, the pharmacologic action of the compounds described herein can be quickly terminated by stopping administration thereof to avoid adverse side effects.

In some aspects, there are described a novel methods of lowering the HR of a patient without a significant lowering of BP, comprising administering intravenously a therapeutic amount of a compound described herein. Examples of HR reduction include 5, 6, 7, 8, 9, 10, 11, 12, 13, 14, 15, 16, 17, 18, 19, 20, 25, 30% (beats per minute), or more compared to the baseline HR prior to compound administration. Further examples of HR reduction include reducing the HR below 100, 95, 90, 85, and 80 BPM. Examples of non-significant lowering of BP include maintaining the same BP as prior to administration of a novel compound described herein. Further examples of non-significant BP lowering include lowering the systolic BP 1, 2, 3, 4, 5, 6, 7, 8, 9, or 10 points (from 1-10 points) and/or lowering the diastolic BP 1, 2, 3, 4, 5, 6,7 8, 9, or 10 points (from 1-10 points).

In some aspects, there are described a novel methods of reducing the sudden increase in HR in a patient during the induction of anesthesia or during surgery without a concomitant clinically significant fall in BP (e.g., a fall as seen with esmolol or IV metoprolol), comprising administering intravenously a therapeutic amount of a compound described herein.

A clinically significant fall in BP is patient dependent. It is a fall in BP that causes undesired symptoms. Examples include a 5, 10, 15, 20 mm or more fall.

In some aspects, there are described a novel methods of controlling HR during CTA (computerized tomography coronary angiography) and/or other cardiac imaging procedures without a concomitant clinically significant fall in BP, comprising administering a therapeutic amount of a compound described herein. In some aspects, the administering is intravenous.

In some aspects, there are described a novel methods of reducing HR without a concomitant clinically significant fall in BP in intensive care patients with sinus tachycardia that utilize excessive O₂, comprising administering a therapeutic amount of a compound described herein. In some aspects, the administering is intravenous.

In some aspects, there are described a novel methods of treating JET arrhythmia in pediatric cardiac surgical patients, comprising administering a therapeutic amount of a compound described herein. In some aspects, the administering is intravenous.

In some aspects, there are described a novel methods of reducing HR intraoperatively, comprising administering intravenously a therapeutic amount of a compound described herein or a stereoisomer or a pharmaceutically acceptable salt thereof, whereby when the drug infusion is terminated the reduction in HR terminates rapidly (rapid “off time”). In some aspects, the administering is intravenous.

In some aspects, there are described a novel methods modulating conduction in the atrioventricular (AV) node, comprising administering intravenously a therapeutic amount of a compound of claim 1 or a stereoisomer or a pharmaceutically acceptable salt thereof.

In some aspects, there are described a novel methods reducing the ventricular response in rapid atrial fibrillation, comprising administering intravenously a therapeutic amount of a compound of claim 1 or a stereoisomer or a pharmaceutically acceptable salt thereof, wherein, a short acting agent would be beneficial if the patient converts to normal sinus rhythm and blood pressure then falls.

In another aspect, route of administration is selected from: intravenous, oral, topical, sublingual, buccal, and through a naso-gastric tube.

In another aspect, a compound described herein is administered intravenously via a bolus followed by an infusion. In some examples, the bolus is from 0.25, 0.5, 0.75, 1, 2, 3, 4, 5, 6, 7, 8, 9, to 10 mg (0.25-10 mg). In some examples, the bolus is from 0.25, 0.5, 0.75, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, to 10 mg/hr (0.25-10 mg/hr). The bolus is typically administered as quickly as possible.

In another aspect, the infusion is 0.1, 0.5, 1, 2, 3, 4, 5, 6, 7, 8, 9, to 10 mg (0.1-10 mg/hr). In some examples, the infusion is from 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, to 10 mg/hr (0.1-10 mg/hr). The infusion is maintained as long as deemed necessary by the medical personnel. Examples include 5, 10, 15, 20, 25, 30, 35, 40, 45, 50, 55, to 60 minutes (5-60 minutes). Additional examples include 1, 2, 3, 4, 5 or more hours (1-5 or more hours).

In another aspect, the blood level reached by the combination of the bolus and infusion is from 0.1, 0.5, 1, 2, 3, 4, 5, 6, 7, 8, 9, 10, 11, 12, 13, 14, 15, 16, 17, 18, 19, to 20 μg/mL mean concentration (0.1-20 μg/mL mean concentration).

In some aspects, there are described novel compounds for use in therapy.

In some aspects, there are described the use of novel compounds for the manufacture of a medicament for the treatment of an indication recited herein.

In another aspect, examples of the molecular weight of the compounds described herein include (a) less than about 600, 650, 700, 750, 800, 850, 900, 950, or 1000 grams per mole; (b) less than about 950 grams per mole; (c) less than about 850 grams per mole; and, (d) less than about 750 grams per mole.

In another aspect, examples of the solubility of the compounds described herein include greater than 50 60, 70, 80, 90, 100, 110, 120, 130, 140, 150, 160, 170, 180, 190, 200, 210, 220, 230, 240, 250, 260, 270, 280, 290, 300, 400, 500, 600, 700, 800, 900 and 1000 μg/mL.

Some aspects may be embodied in other specific forms without departing from the spirit or essential attributes thereof. This invention encompasses all combinations of aspects described herein. It is understood that any and all aspects described herein may be taken in conjunction with any other embodiment or embodiments to describe additional embodiments. It is also to be understood that each individual element of the embodiments is intended to be taken individually as its own independent embodiment. Furthermore, any element of an embodiment is meant to be combined with any and all other elements from any embodiment to describe an additional embodiment.

### Definitions

The examples provided in the definitions present in this application are non-inclusive unless otherwise stated. They include but are not limited to the recited examples.

A compound or compounds, as used herein, includes, where appropriate, stereoisomers and/or pharmaceutically acceptable salts thereof.

The compounds described herein may have asymmetric centers, geometric centers (e.g., double bond), or both. All chiral, diastereomeric, racemic forms and all geometric isomeric forms of a structure are intended, unless the specific stereochemistry or isomeric form is specifically indicated. Compounds described herein containing an asymmetrically substituted atom may be isolated in optically active or racemic forms. It is well known in the art how to prepare optically active forms, such as by resolution of racemic forms, by synthesis from optically active starting materials, or through use of chiral auxiliaries. Geometric isomers of olefins, C═N double bonds, or other types of double bonds may be present in the compounds described herein, and all such stable isomers are included in some aspects. Specifically, cis and trans geometric isomers of the compounds described herein may also exist and may be isolated as a mixture of isomers or as separated isomeric forms. All processes used to prepare compounds described herein and intermediates made therein are part of some aspects. All tautomers of shown or described compounds are also considered to be part of some aspects.

Compounds with at least two stereocenters are referred to as being present in “diastereomeric excess” (de). The % de is determined by the following formula:

% de=(% A−% B)/(% A+% B)*100

wherein A=desired diastereomer, B=undesired diastereomer, and %=mole fraction. For example, 60% de refers to 80% of the desired diastereomer and 20% of the undesired diastereomer(s). Examples include 60, 70, 80, 90, 91, 92, 93, 94, 95, 96, 97, 98, to 99% de.

Some aspects includes all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The term “substituted” means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties.

“Stable” means that the compound is suitable for pharmaceutical use.

Some aspects covers stable compounds and thus avoids, unless otherwise specified, the following bond types: heteroatom-halogen, N—S, O—S, O—O, and S—S.

“Alkyl” includes both branched and straight-chain saturated aliphatic hydrocarbon groups having the specified number of carbon atoms. C₁₋₆ alkyl, for example, includes C₁, C₂, C₃, C₄, C₅, and C₆ alkyl groups. Examples of alkyl include methyl, ethyl, n-propyl, i-propyl, n-butyl, s-butyl, t-butyl, n-pentyl, and s-pentyl.

When an “ene” terminates a group it indicates the group is attached to two other groups. For example, methylene refers to a —CH₂-moiety.

“Alkenyl” includes the specified number of hydrocarbon atoms in either straight or branched configuration with one or more unsaturated carbon-carbon bonds that may occur in any stable point along the chain, such as ethenyl and propenyl. C₂₋₆ alkenyl includes C₂, C₃, C₄, C₅, and C₆ alkenyl groups.

“Alkynyl” includes the specified number of hydrocarbon atoms in either straight or branched configuration with one or more triple carbon-carbon bonds that may occur in any stable point along the chain, such as ethynyl and propynyl. C₂₋₆ alkynyl includes C₂, C₃, C₄, C₅, and C₆ alkynyl groups.

“Cycloalkyl” includes the specified number of hydrocarbon atoms in a saturated ring, such as cyclopropyl, cyclobutyl, cyclopentyl, cyclohexyl, cycloheptyl, and cyclooctyl. C₃₋₈ cycloalkyl includes C₃, C₄, C₅, C₆, C₇, and C₈ cycloalkyl groups.

“Halo” or “halogen” refers to fluoro, chloro, bromo, and iodo.

“Counterion” is used to represent a small, negatively charged species, such as chloride, bromide, hydroxide, acetate, and sulfate.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting its development; and/or (c) relieving the disease-state, e.g., causing regression of the disease state until a desired endpoint is reached. Treating also includes the amelioration of a symptom of a disease, wherein such amelioration may or may not be directly affecting the disease (e.g., cause, transmission, expression, etc.).

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

The pharmaceutically acceptable salts described herein can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are useful. Lists of suitable salts are found in Remington's Pharmaceutical Sciences, 18th ed., Mack Publishing Company, Easton, PA, 1990, p 1445, the disclosure of which is hereby incorporated by reference.

“Therapeutically effective amount” includes an amount of a compound described herein that is effective when administered alone or in combination to an indication listed herein. “Therapeutically effective amount” also includes an amount of the combination of compounds claimed that is effective to treat the desired indication. The combination of compounds can be a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased effect, or some other beneficial effect of the combination compared with the individual components.

### Formulations and Dosages

The compounds described herein can be formulated as pharmaceutical compositions and administered to a human patient in a variety of forms adapted to the chosen route of administration, e.g., orally, parenterally, by intravenous (e.g., continuously or bolus), intrathecal, intramuscular, topical, intradermal, intraperitoneal, intraocular, inhalation or subcutaneous routes. Exemplary pharmaceutical compositions are disclosed in “Remington: The Science and Practice of Pharmacy, ” A. Gennaro, ed., 20th edition, Lippincott, Williams & Wilkins, Philadelphia, PA.

Thus, the present compounds may be systemically administered, e.g., intravenously, in combination with a pharmaceutically acceptable carrier/excipient such as an inert diluent. The amount of active compound in such therapeutically useful compositions is such that an effective dosage level will be obtained.

The amount of the compound described herein or an active salt or derivative thereof, required for use in treatment will vary not only with the compound or salt selected but also with the route of administration, the nature of the condition being treated, and the age and condition of the patient, and will ultimately be at the discretion of the attendant physician or clinician.

The compounds described herein can be conveniently administered in unit dosage form, e.g., vials or pre-filled syringes, etc., containing (a) about 100-100 mg, (b) about 200-800 mg, and (c) about 400-600 mg of active ingredient per unit dosage form.

The compounds described herein can be administered to achieve peak plasma concentrations of the active compound of about 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, to 3.5 ng/mL. These concentrations may be achieved, for example, by the intravenous injection (e.g., continuously or bolus) of a 0.005-0.5% solution of the active ingredient.

When a compound described herein is administered in combination with another agent or agents (e.g., co-administered), then the compound described herein and other agent can be administered simultaneously or in any order. They can be administered as a single pharmaceutical composition or as separate compositions. The administration of the compound described herein can be prior to the other agent(s), within minutes thereof, or up to hours (e.g., 24 or 48) or even days after the administration of the other agent(s). For example, the administration of the compound described herein can be within about 24 hours or within about 12 hours.

The compounds described herein may also be administered intravenously (e.g., continuously or bolus) or intraperitoneally by infusion or injection. Solutions of the compounds described herein or their salts can be prepared in water, optionally mixed with a nontoxic surfactant. For solubility purposes the compound(s) may also be solubilized in a lipid emulsion or administered in liposomes. Dispersions can also be prepared in glycerol, liquid polyethylene glycols, triacetin, and mixtures thereof and in oils. Under ordinary conditions of storage and use, these preparations contain a preservative to prevent the growth of microorganisms. The composition can be administered as an emulsion or a nanoparticle.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or using surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions. Sterilization can also be performed by rapid heating and cooling.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings or sprayed onto the affected area using pump-type or aerosol sprayers.

The desired dose of the compounds described herein may conveniently be presented in a single dose or as divided doses administered at appropriate intervals, for example, as two, three, four or more sub-doses per day. The sub-dose itself may be further divided, e.g., into a number of discrete loosely spaced administrations.

### SYNTHESIS

The compounds described herein can be prepared in a number of ways known to one skilled in the art of organic synthesis (e.g., see U.S. Pat. No. 7,176,197, EP 2097383 and EP 0534859). The compounds described herein can be synthesized using the methods described below, together with synthetic methods known in the art of synthetic organic chemistry, or by variations thereon as appreciated by those skilled in the art. Preferred methods include, but are not limited to, those described below. The reactions are performed in a solvent appropriate to the reagents and materials employed and suitable for the transformations being affected. It will be understood by those skilled in the art of organic synthesis that the functionality present on the molecule should be consistent with the transformations proposed. This will sometimes require a judgment to modify the order of the synthetic steps or to select one process scheme over another in order to obtain a desired compound. It will also be recognized that another major consideration in the planning of any synthetic route in this field is the judicious choice of the protecting group used for protection of the reactive functional groups present in the compounds described herein. An authoritative account describing the many alternatives to the trained practitioner is Greene and Wuts (Protective Groups In Organic Synthesis, Wiley and Sons, 1991). All references cited herein are hereby incorporated in their entirety herein by reference.

One stereoisomer of a compound described herein may be more potent than its counterpart(s). Thus, stereoisomers are included in some aspects. When required, separation of the racemic material can be achieved by HPLC using a chiral column or by a resolution using a resolving agent such as described in Wilen, S. H. Tables of Resolving Agents and Optical Resolutions 1972, 308 or using enantiomerically pure acids and bases. A chiral compound described herein may also be directly synthesized using a chiral catalyst or a chiral ligand, e.g., Jacobsen, E. Acc. Chem. Res. 2000, 33, 421-431 or using other enantio-and diastereo-selective reactions and reagents known to one skilled in the art of asymmetric synthesis.

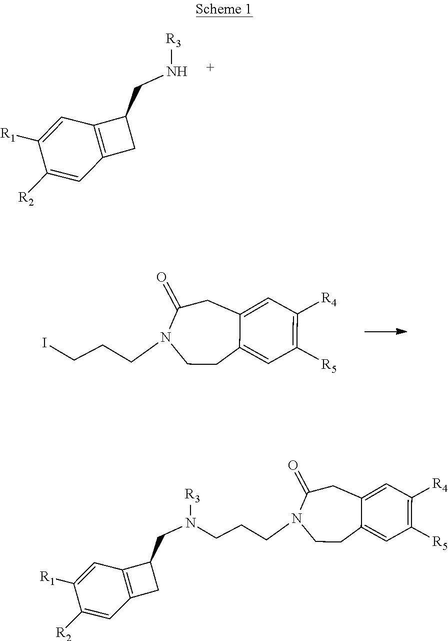

Scheme 1 shows the preparation of ivabradine analogs with functionality to confer ultra-short activity to the molecules due to their propensity for rapid esterase hydrolysis to metabolites with greatly diminished, or absence of, parent compound activity. The approach to the synthesis of analogs of ivabradine and related compounds has been duly demonstrated (see, for example, CN 101544605). By altering R₁-R₅, various esterified versions of ivabradine can be made.

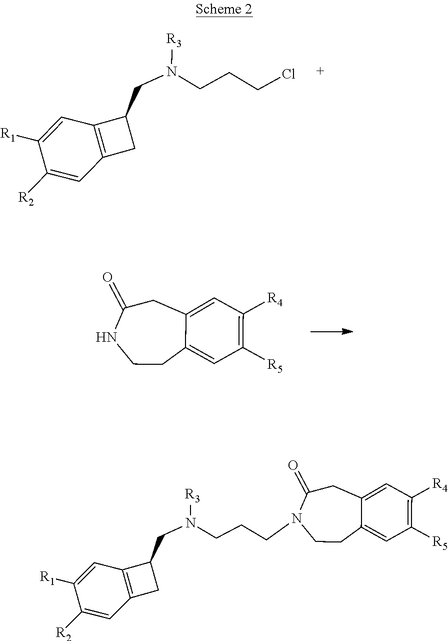

Scheme 2 shows an alternative preparation of ivabradine analogs. The approach has been duly demonstrated (see, for example, EP2097383B1). By altering R₁—R₅, various esterified versions of ivabradine can be made.

Other features of the aspects will become apparent in the course of the following descriptions of exemplary embodiments that are given for illustration of the aspects and are not intended to be limiting thereof.

### EXAMPLES

The examples in the tables below can be prepared according to the methods of the scheme numbers provided for each example.

TABLE 1

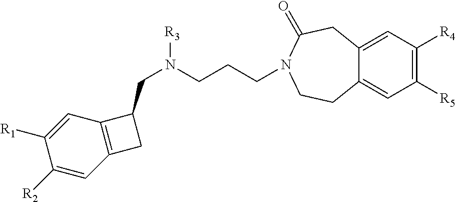

| Ex. # | R₁ | R₂ | R₃ | R₄ | R₅ |
|---|---|---|---|---|---|
| 1. | OCH₃ | OCH₃ | CH₃ | OCH₃ | CO₂H |
| 2. | OCH₃ | OCH₃ | CH₃ | OCH₃ | CO₂CH₃ |
| 3. | OCH₃ | OCH₃ | CH₃ | CO₂H | OCH₃ |
| 4. | OCH₃ | OCH₃ | CH₃ | CO₂CH₃ | OCH₃ |
| 5. | OCH₃ | OCH₃ | CO₂H | OCH₃ | OCH₃ |
| 6. | OCH₃ | OCH₃ | CO₂CH₃ | OCH₃ | OCH₃ |
| 7. | OCH₃ | CO₂H | CH₃ | OCH₃ | OCH₃ |
| 8. | OCH₃ | CO₂CH₃ | CH₃ | OCH₃ | OCH₃ |
| 9. | CO₂H | OCH₃ | CH₃ | OCH₃ | OCH₃ |
| 10. | CO₂CH₃ | OCH₃ | CH₃ | OCH₃ | OCH₃ |
| 11. | OCH₃ | OCH₃ | CH₃ | CO₂H | CO₂H |
| 12. | OCH₃ | OCH₃ | CH₃ | CO₂CH₃ | CO₂CH₃ |
| 13. | OCH₃ | OCH₃ | CO₂H | CO₂H | CO₂H |
| 14. | OCH₃ | OCH₃ | CO₂CH₃ | CO₂CH₃ | CO₂CH₃ |
| 15 | OCH₃ | CO₂H | CH₃ | CO₂H | CO₂H |
| 16. | OCH₃ | CO₂CH₃ | CH₃ | CO₂CH₃ | CO₂CH₃ |
| 17. | CO₂H | OCH₃ | CH₃ | CO₂H | CO₂H |
| 18. | CO₂CH₃ | OCH₃ | CH₃ | CO₂CH₃ | CO₂CH₃ |
| 19. | OCH₃ | OCH₃ | CO₂H | CO₂H | OCH₃ |
| 20. | OCH₃ | OCH₃ | CO₂CH₃ | CO₂CH₃ | OCH₃ |
| 21. | OCH₃ | CO₂H | CO₂H | CO₂H | OCH₃ |
| 22. | OCH₃ | CO₂CH₃ | CO₂CH₃ | CO₂CH₃ | OCH₃ |
| 23. | CO₂H | OCH₃ | CO₂H | CO₂H | OCH₃ |
| 24. | CO₂CH₃ | OCH₃ | CO₂CH₃ | CO₂CH₃ | OCH₃ |
| 25. | OCH₃ | CO₂H | CO₂H | OCH₃ | OCH₃ |
| 26. | OCH₃ | CO₂CH₃ | CO₂CH₃ | OCH₃ | OCH₃ |
| 27. | CO₂H | CO₂H | CO₂H | OCH₃ | OCH₃ |
| 28. | CO₂CH₃ | CO₂CH₃ | CO₂CH₃ | OCH₃ | OCH₃ |
| 29. | OCH₃ | OCH₃ | CO₂H | OCH₃ | CO₂H |
| 30. | OCH₃ | OCH₃ | CO₂CH₃ | OCH₃ | CO₂CH₃ |
| 31. | OCH₃ | CO₂H | CO₂H | OCH₃ | CO₂H |
| 32. | OCH₃ | CO₂CH₃ | CO₂CH₃ | OCH₃ | CO₂CH₃ |
| 33. | CO₂H | OCH₃ | CO₂H | OCH₃ | CO₂H |
| 34. | CO₂CH₃ | OCH₃ | CO₂CH₃ | OCH₃ | CO₂CH₃ |
| 35. | OCH₃ | CO₂H | CH₃ | OCH₃ | CO₂H |
| 36. | OCH₃ | CO₂CH₃ | CH₃ | OCH₃ | CO₂CH₃ |
| 37. | CO₂H | CO₂H | CH₃ | OCH₃ | CO₂H |
| 38. | CO₂CH₃ | CO₂CH₃ | CH₃ | OCH₃ | CO₂CH₃ |
| 39. | CO₂H | OCH₃ | CH₃ | OCH₃ | CO₂H |
| 40. | CO₂CH₃ | OCH₃ | CH₃ | OCH₃ | CO₂CH₃ |

Numerous modifications and variations of the aspects described herein are possible in light of the above teachings. It is therefore to be understood that within the scope of the appended claims, the aspects may be practiced otherwise than as specifically described herein.

What is claimed is:

1. A compound of Formula I or a stereoisomer or pharmaceutically acceptable salt thereof: 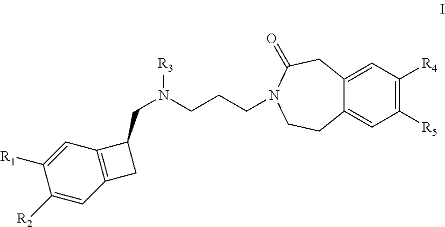
wherein:
at least one of R₁, R₂, R₃, R₄, and R₅ is —CO₂R_{A};
the remainder of R₁, R₂, R₄, and R₅ are independently selected from: OH, OC₁₋₆ alkyl, OCH₂C₆H₅, and OCOCH₃;
R_{A} is independently selected from: H, C₁₋₆ alkyl, C₃₋₆ alkenyl, and C₃₋₆ alkynyl; and,
R₃, if not CO₂R_{A}, is selected from: H, C₁₋₆ alkyl, —CH₂C₆H₅, and —COCH₃.

2. A compound of claim 1, wherein:
at least one of R₁, R₂, R₃, R₄, and R₅ is —CO₂R_{A};
the remainder of R₁, R₂, R₄, and R₅ are OCH₃;
R_{A} is independently selected from: H and CH₃;
R₃, if not CO₂R_{A}, is CH₃.

3. A compound of claim 1, wherein:
R_{A} is independently CH₃.

4. A compound of claim 1, wherein:
at least two of R₁, R₂, R₃, R₄, and R₅ are —CO₂R_{A};
the remainder of R₁, R₂, R₄, and R₅ are OCH₃;
R_{A} is independently selected from: H and CH₃;
R₃, if not CO₂R_{A}, is CH₃.

5. A compound of claim 1, wherein:
at least three of R₁, R₂, R₃, R₄, and R₅ are —CO₂R_{A};
the remainder of R₁, R₂, R₄, and R₅ are OCH₃;
R_{A} is independently selected from: H and CH₃;
R₃, if not CO₂R_{A}, is CH₃.

6. A compound of claim 1, wherein:
at least four of R₁, R₂, R₃, R₄, and R₅ are —CO₂R_{A};
the remainder of R₁, R₂, R₄, and R₅ are OCH₃;
R_{A} is independently selected from: H and CH₃;
R₃, if not CO₂R_{A}, is CH₃.

7. A compound of claim 1, wherein:
R₁, R₂, R₃, R₄, and R₅ are all —CO₂R_{A}; and,
R_{A} is independently selected from: H and CH₃.

8. A compound of claim 1, wherein the compound is of Formula IA or a stereoisomer or a pharmaceutically acceptable salt thereof: 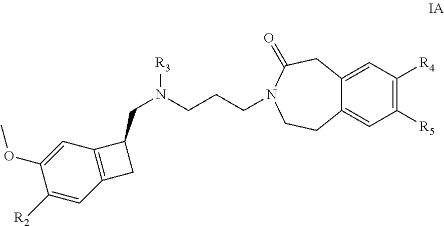
at least one of R₂, R₃, R₄, and R₅ is —CO₂R_{A};
the remainder of R₂, R₄, and R₅ are OCH₃;
R_{A} is independently selected from: H and CH₃;
R₃, if not CO₂R_{A}, is CH₃.

9. A compound of claim 1, wherein the compound is of Formula IA₁ or a stereoisomer or a pharmaceutically acceptable salt thereof: 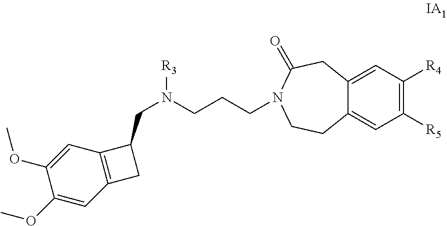
at least one of R₃, R₄, and R₅ is —CO₂R_{A};
the remainder of R₄, and R₅ are OCH₃;
R_{A} is independently selected from: H and CH₃;
R₃, if not CO₂R_{A}, is CH₃.

10. A compound of claim 1, wherein the compound is of Formula IB or a stereoisomer or a pharmaceutically acceptable salt thereof: 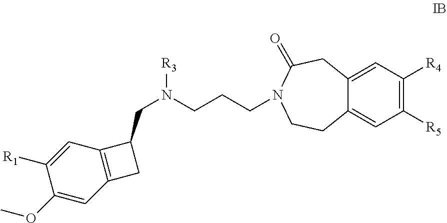
at least one of R₁, R₃, R₄, and R₅ is —CO₂R_{A};
the remainder of R₁, R₄, and R₅ are OCH₃;
R_{A} is independently selected from: H and CH₃;
R₃, if not CO₂R_{A}, is CH₃.

11. A compound of claim 1, wherein the compound is of Formula IC or a stereoisomer or a pharmaceutically acceptable salt thereof: 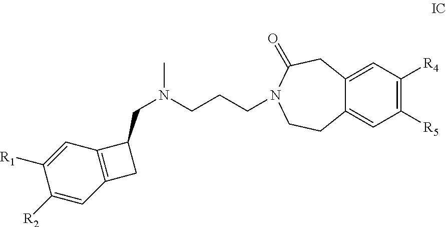
at least one of R₁, R₂, R₄, and R₅ is —CO₂R_{A};
the remainder of R₁, R₂, R₄, and R₅ are OCH₃; and,
R_{A} is independently selected from: H and CH₃.

12. A compound of claim 1, wherein the compound is of Formula ID or a stereoisomer or a pharmaceutically acceptable salt thereof: 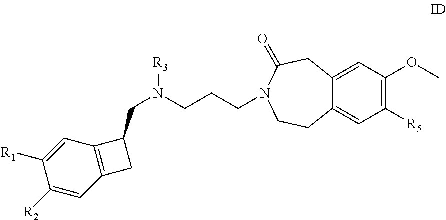
at least one of R₁, R₂, R₃, and R₅ is —CO₂R_{A};
the remainder of R₁, R₂, and R₅ are OCH₃;
R_{A} is independently selected from: H and CH₃;
R₃, if not CO₂R_{A}, is CH₃.

13. A compound of claim 1, wherein the compound is of Formula ID₁ or a stereoisomer or a pharmaceutically acceptable salt thereof: 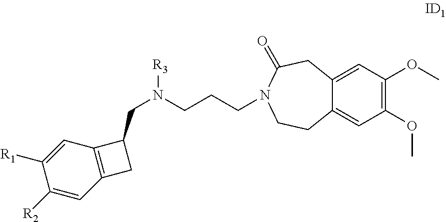
at least one of R₁, R₂, and R₃ is —CO₂R_{A};
the remainder of R₁ and R₂ are OCH₃;
R_{A} is independently selected from: H and CH₃;
R₃, if not CO₂R_{A}, is CH₃.

14. A compound of claim 1, wherein the compound is of Formula IE or a stereoisomer or a pharmaceutically acceptable salt thereof: 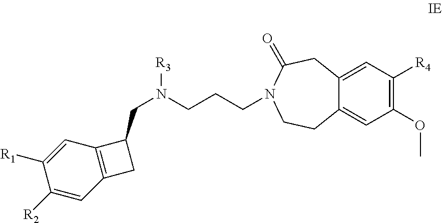
at least one of R₁, R₂, R₃, and R₄ is —CO₂R_{A};
the remainder of R₁, R₂, and R₄ are OCH₃;
R_{A} is independently selected from: H and CH₃;
R₃, if not CO₂R_{A}, is CH₃.

15. A pharmaceutical composition, comprising: a therapeutically effective amount of a compound of claim 1 or a stereoisomer or a pharmaceutically acceptable salt thereof and a pharmaceutically acceptable carrier.

16. A method of lowering the heart rate (HR) of a patient without a significant lowering of BP, comprising administering intravenously a therapeutic amount of a compound of claim 1 or a stereoisomer or a pharmaceutically acceptable salt thereof.

17. A method of reducing the sudden increase in heart rate (HR) in a patient during the induction of anesthesia or during surgery without a concomitant clinically significant fall in BP, comprising administering intravenously a therapeutic amount of a compound of claim 1 or a stereoisomer or a pharmaceutically acceptable salt thereof.

18. A method of controlling heart rate (HR) during CTA (computerized tomography coronary angiography) and/or other cardiac imaging procedures without a concomitant clinically significant fall in BP, comprising administering intravenously a therapeutic amount of a compound of claim 1 or a stereoisomer or a pharmaceutically acceptable salt thereof.

19. A method of reducing heart rate (HR) without a concomitant clinically significant fall in BP in intensive care patients with sinus tachycardia that utilize excessive O₂, comprising administering intravenously a therapeutic amount of a compound of claim 1 or a stereoisomer or a pharmaceutically acceptable salt thereof.

20. A method of treating junctural ectopic tachycardia (JET) arrhythmia in pediatric cardiac surgical patients, comprising administering intravenously a therapeutic amount of a compound of claim 1 or a stereoisomer or a pharmaceutically acceptable salt thereof.

21. A method of reducing heart rate (HR) intraoperatively, comprising administering intravenously a therapeutic amount of a compound of claim 1 or a stereoisomer or a pharmaceutically acceptable salt thereof, whereby when the drug infusion is terminated the reduction in HR terminates rapidly (rapid “off time”).

22. A method of modulating conduction in the atrioventricular (AV) node, comprising administering intravenously a therapeutic amount of a compound of claim 1 or a stereoisomer or a pharmaceutically acceptable salt thereof.

23. A method of reducing the ventricular response in rapid atrial fibrillation, comprising administering intravenously a therapeutic amount of a compound of claim 1 or a stereoisomer or a pharmaceutically acceptable salt thereof, wherein, a short acting agent would be beneficial if the patient converts to normal sinus rhythm and blood pressure then falls.
